(19) United States
(12) Patent Application Publication
(10) Pub. No.: US 2026/0301930 A1
(43) Pub. Date: Oct. 1, 2026

(54) EXAMINATION WORK SUPPORT APPARATUS, EXAMINATION WORK SUPPORT SYSTEM, AND EXAMINATION WORK SUPPORT METHOD

(71) Applicant: Konica Minolta, Inc., Tokyo (JP)

(72) Inventors: Tatsuya Takagi, Tokyo (JP); Kenichi Yanagisawa, Tokyo (JP); Kazuhiro Kojima, Osaka (JP); Ryouhei Kikuchi, Tokyo (JP); Atsushi Taneda, Tokyo (JP)

(73) Assignee: Konica Minolta, Inc., Tokyo (JP)

(21) Appl. No.: 19/634,612

(22) Filed: Mar. 31, 2026

(30) Foreign Application Priority Data
Mar. 31, 2025 (JP) 2025-057999

(51) Int. Cl. G16H 40/20; G06T 7/70; G06V 40/20; G16H 10/60

(52) U.S. Cl. CPC G16H 40/20; G06T 7/70; G06V 40/20; G16H 10/60; G06T 2207/30196

(57) ABSTRACT

An examination work support apparatus includes a hardware processor that acquires medical information, sensor information acquirable in real time, and prior information, extracts, based on the acquired information, one or more of patient position information on a patient, patient attribute information on the patient, and medical professional position information on a medical professional, determines, based on the acquired information and the extracted information, support information on one or more of an examination order number for the patient, an examination room, and a duty assigned to the medical professional, and causes a notifier to notify the determined support information.

### CROSS-REFERENCE TO RELATED APPLICATION

The entire disclosure of Japanese Patent Application No. 2025-057999, filed on Mar. 31, 2025, including description, claims, drawings and abstract is incorporated herein by reference.

### BACKGROUND OF THE INVENTION

### Technical Field

The present disclosure relates to an examination work support apparatus, an examination work support system, and an examination work support method.

### Description of Related Art

There has been a demand for improving the efficiency of examination work in hospitals.

For example, in radiological examination work, with an increase in elderly patients, the number of examinations has been increasing, and they have been getting more difficult. However, it is expected that the number of radiology examination technicians follows changes in the working-age population and accordingly gradually decreases, and, together with improvements in the labor environment, such as work-style reforms, it becomes difficult to secure personnel resources. Accordingly, there is a need to improve the efficiency of radiological examination work.

Japanese Unexamined Patent Application Publication No. 2008-125734 describes displaying a congestion status based on patient position information.

International Publication No. 2018/193985 describes acquiring patient position information, associating a medical care process with each patient, and displaying a progress status of the medical care process for each patient.

### SUMMARY OF THE INVENTION

Japanese Unexamined Patent Application Publication No. 2008-125734 and International Publication No. 2018/193985 merely display the congestion status or the progress status, and do not necessarily improve the efficiency of examination work.

Accordingly, an object of the present disclosure is to more effectively improve the efficiency of examination work.

To achieve at least one of the abovementioned objects, according to an aspect of the present disclosure, an examination work support apparatus reflecting one aspect of the present disclosure includes a hardware processor that
acquires medical information, sensor information acquirable in real time, and prior information,
extracts, based on the acquired information, one or more of patient position information on a patient, patient attribute information on the patient, and medical professional position information on a medical professional,
determines, based on the acquired information and the extracted information, support information on one or more of an examination order number for the patient, an examination room, and a duty assigned to the medical professional, and
causes a notifier to notify the determined support information.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a block diagram illustrating a configuration of an examination work support system;

FIG. 2 is an illustrative diagram of a radiology department;

FIG. 3 is a flowchart of a support process;

FIG. 4 illustrates an example of prior information;

FIG. 5 is an example of a patient assignment result;

FIG. 6 is an illustrative diagram of a support screen; and

FIG. 7 is a flowchart of a support process.

### DETAILED DESCRIPTION

Hereinafter, one or more embodiments of the present disclosure will be described with reference to the drawings. However, the scope of the invention is not limited to the disclosed embodiments.

### Examination Work Support System 100

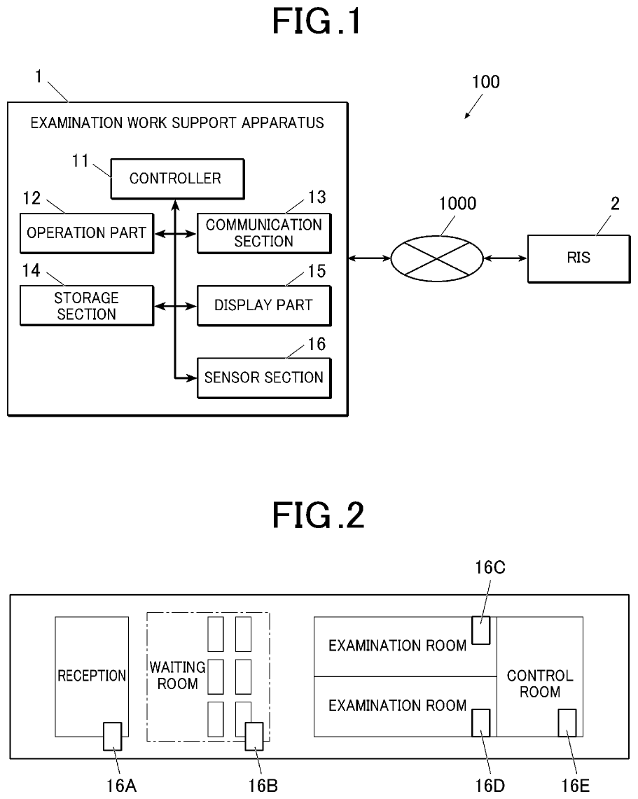

First, the configuration of an examination work support system 100 will be described with reference to FIG. 1. As illustrated in FIG. 1, the examination work support system 100 includes an examination work support apparatus 1 and a radiology information system (RIS) 2 that are connected via a communication network 1000 such as a local area network (LAN).

The examination work support apparatus 1 is an apparatus for supporting examination work performed by a medical professional. The examination work support apparatus 1 includes a sensor section 16, which will be described later, and supports examination work performed by a medical professional by using real-time information (sensor information) transmitted from the sensor section 16.

In the following description, the medical professional is described as a radiology technician; however, the medical professional is not limited thereto, and may be a technician in ophthalmology, otorhinolaryngology, or the like.

The RIS 2 is an apparatus for managing medical information on a patient. The medical information includes examination information such as an examination name and examination details.

The medical information is described as information stored in a radiology work management system (RIS 2); however, the medical information is not limited thereto, and may be stored in various information processing apparatuses.

### Radiology Department

An illustrative diagram of a radiology department will now be described with reference to FIG. 2.

The radiology department includes rooms such as a reception area, a waiting room, an examination room, and a control room. The control room refers to a room in which a technician operates a radiographic imaging apparatus in the examination room.

As illustrated in FIG. 2, it is assumed that sensor sections 16 (16A, 16B, 16C, 16D, 16E) are disposed in respective rooms in the radiology department.

Note that the numbers of reception rooms, waiting rooms, examination rooms, and control rooms may each be two or more. In addition, each sensor section 16 (16A, 16B, 16C, 16D, 16E) may include a plurality of sensors.

### Examination Work Support Apparatus 1

Next, the configuration of the examination work support apparatus 1 will be described with reference to FIG. 1. As illustrated in FIG. 1, the examination work support apparatus 1 includes a controller 11 (hardware processor), an operation part 12, a communication section 13, a storage section 14, a display part 15, and a sensor section.

The controller 11 includes a CPU (central processing unit), a RAM (random access memory), and the like. The CPU of the controller 11 reads various programs stored in the storage section 14, loads the programs into the RAM, executes various processes in accordance with the loaded programs, and controls operations of the respective components of the examination work support apparatus 1.

The controller 11 functions as an acquisition section configured to acquire medical information, sensor information acquirable in real time, and prior information.

Here, the medical information includes patient information such as the patient's name and age, examination information (modality, body part), and the like.

The sensor information is information for detecting a patient or a technician and is, for example, image data.

The prior information includes various types of information on a medical professional.

For example, the prior information includes standard examination time information for each attribute, each examination, and each medical professional, as described below. Note that, as described below, when the attribute information is extracted by the controller 11, the prior information need not include the attribute information.

In addition, for example, the prior information includes performable examination information for each examination room and for each medical professional.

The controller 11 functions as an extraction section configured to extract one or more of the patient position information, the patient attribute information, and the medical professional position information, based on the information acquired by the acquisition section.

The attribute information is information indicating an attribute of a patient. For example, the attribute information includes information indicating that the patient requires a wheelchair or a cane, information indicating that the patient is elderly, and the like. As for extraction of the attribute information, various methods, such as the one disclosed in Japanese Unexamined Patent Publication No. 2024-078008, may be used.

The controller 11 functions as a determination section configured to determine, based on the information acquired by the acquisition section and the information extracted by the extraction section, support information on one or more of an examination order number for a patient, an examination room, and a duty assigned to a medical professional.

For example, as described below, the controller 11 functions as a determination section configured to determine the support information based on a viewpoint of minimization of a patient's waiting time and/or maximization of the number of examinations per unit time.

The controller 11 functions as a notification controller configured to cause a notifier to notify the support information determined by the determination section.

The controller 11 functions as an assessment section configured to assess the degree of congestion (congestion degree) of the waiting room based on the sensor information.

Note that the controller 11 may, based on the congestion degree, switch necessity and unnecessity of the process as the determination section or the level of the process as the determination section. For example, if the congestion degree is greater than a predetermined value, the controller 11 may perform the process as the determination section and/or increase the level of the process as the determination section. Here, the level of the process refers to “the degree of minimization of the patient's waiting time and/or maximization of the number of examinations per unit time”. The controller 11 lowers the degree of minimization of the waiting time to provide a technician with discretion when the waiting room is not crowded, and raises the degree of minimization of the waiting time when the waiting room is crowded.

The operation part 12 may include a keyboard provided with cursor keys, numeric input keys, various function keys, and the like; a pointing device such as a mouse; and a touch screen laminated on the surface of the display part 15. The operation part 12 is configured to be operated by an operator. Also, the operation part 12 outputs, to the controller 11, various signals based on operations performed by the operator.

The communication section 13 is capable of transmitting and receiving various signals and various data to and from other devices and the like that are communicatively connected via the communication network 1000.

The storage section 14 is constituted by a nonvolatile semiconductor memory, a hard disk, or the like, and stores various programs to be executed by the controller 11, parameters required to execute the programs, and various data.

For example, the storage section 14 stores the prior information and the like.

The display part 15 is constituted by a monitor such as a liquid crystal display (LCD), and displays various screens and the like in accordance with instructions of display signals input from the controller 11. The display part 15 may further include a speaker configured to output sound, a lamp that blinks, or the like.

The display part 15 functions as the notifier.

The sensor section 16 is configured to acquire various types of information on a patient or a technician.

For example, the sensor section 16 is an optical camera configured to acquire a visible image (optical camera image) as the sensor information. The visible image may be a still image or a moving image.

### Support Process: Case 1

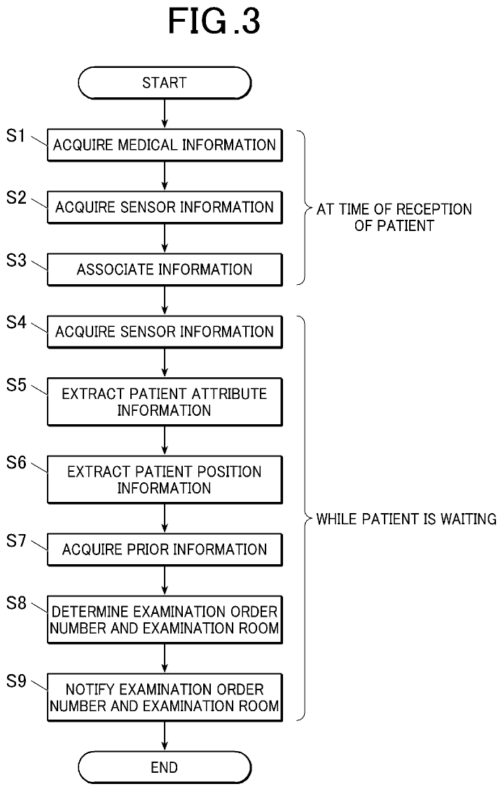

Next, a support process in the examination work support apparatus 1 will be described with reference to FIG. 3.

### (At Time of Reception of Patient)

First, the controller 11 acquires medical information from the RIS 2 via the communication section 13 (Step S1; acquisition step). Specifically, the controller 11 acquires medical information such as a name, an age, and examination information (modality, body part) of a patient.

Next, the controller 11 acquires sensor information from the sensor section 16 (Step S2; acquisition step). Specifically, the controller 11 acquires, from the sensor section 16A disposed at a reception area, an optical camera image at the time of reception of the patient.

Next, the controller 11 associates the medical information with the sensor information (Step S3). Specifically, the controller 11 associates the optical camera image at the time of reception of the patient with the medical information on the patient received/registered at the same time.

### (While Patient is Waiting)

Next, the controller 11 acquires sensor information from the sensor section 16 (Step S4: acquisition step) and identifies the patient. Specifically, the controller 11 acquires, from the sensor section 16B disposed in the waiting room, an optical camera image obtained while the patient is waiting and identifies the patient.

Next, the controller 11 extracts patient attribute information on the patient from the sensor information (Step S5; extraction step). Specifically, the controller 11 extracts information indicating that the patient requires a wheelchair or a cane, and/or information indicating that the patient is elderly.

Next, the controller 11 extracts patient position information on the patient from the sensor information (Step S6; extraction step). Specifically, the controller 11 determines where the patient is, from the sensor section 16B disposed in the waiting room to acquire the position information. For example, in a case where there are two or more waiting rooms, the controller 11 can determine where in which waiting room the patient is.

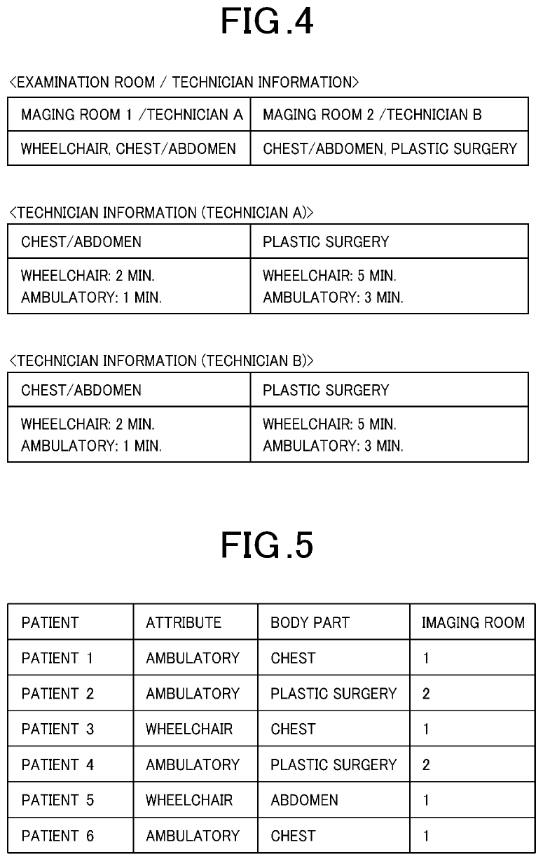

Next, the controller 11 acquires prior information from the storage section 14 (Step S7; acquisition step). Specifically, the controller 11 acquires the prior information as illustrated in FIG. 4.

The prior information illustrated in FIG. 4 will be described hereinafter. The “Examination Room/Technician Information” is information indicating an examination(s) performable by a combination of an examination room and a technician. The “Technician Information (Technician A)” and the “Technician Information (Technician B)” are each information indicating times required for a technician to perform examinations.

Next, the controller 11 determines, as support information, an examination order number and an examination room in which an examination is to be performed, for each patient (Step S8; determination step). Specifically, the controller 11 determines the examination order number and the examination room for each patient by determining patient assignment as illustrated in FIG. 5.

The assignment result of patients illustrated in FIG. 5 will be described hereinafter. The patient assignment illustrated in FIG. 5 makes the total examination time for an examination room 1 be 6 minutes and the total examination time for an examination room 2 be 6 minutes, which results in the earliest examination end time. Note that, as a precondition, for example, a patient with a wheelchair (attribute) needs to be assigned to an examination room in which an examination can be performed for a wheelchair user. Further, in the case of an ambulatory person (attribute), the patient may be assigned to the closest examination room based on the extracted patient position information on the patient. Further, for a patient who is an elderly person (attribute), it may be taken into consideration that changing clothes or the like may take time.

Note that the determination method may involve trying all assignment patterns to make the determination, or learning an optimal combination in advance and performing inference processing using machine learning.

If examination reception times of patients 1 to 6 are different from each other, an examination end time minus an examination reception time for each patient is defined as a waiting time of the patient, and the patients are assigned such that the total sum of the waiting times becomes the minimum.

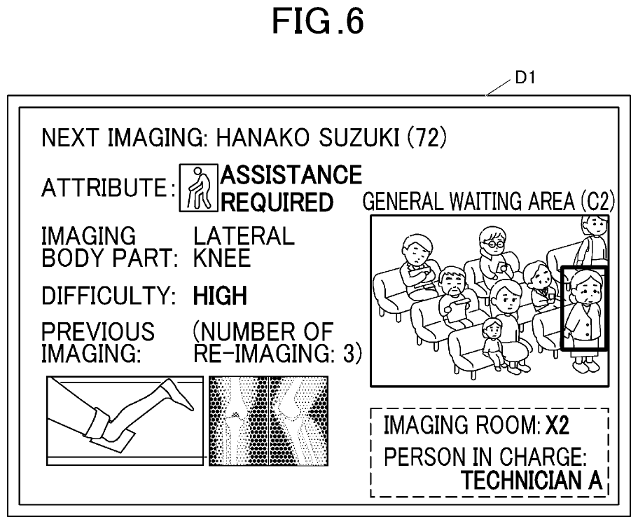

Next, the controller 11 causes the display part 15 to notify the examination order number and the examination room (Step S9; notification control step), and ends the support process. Specifically, the controller 11 causes the display part 15 to display a support screen D1 illustrated in FIG. 6.

The support screen DI illustrated in FIG. 6 will now be described. On the support screen D1 illustrated in FIG. 6, a patient name, extracted attribute information, and an examination room (and a technician of the examination room) are displayed in order of examinations.

In this manner, the efficiency of the examination work can be improved more effectively.

More specifically, by optimizing the patient assignment, efficiently-performable-examination-and-patient combinations can be selected for respective examination-room-and-technician combinations. In addition, since the assignment is determined with the attribute information taken into account, a decrease in the operating rate of an examination room due to imaging preparation, patient reception, patient cloth change, and the like can be prevented, thereby improving examination efficiency.

### Support Process: Case 2

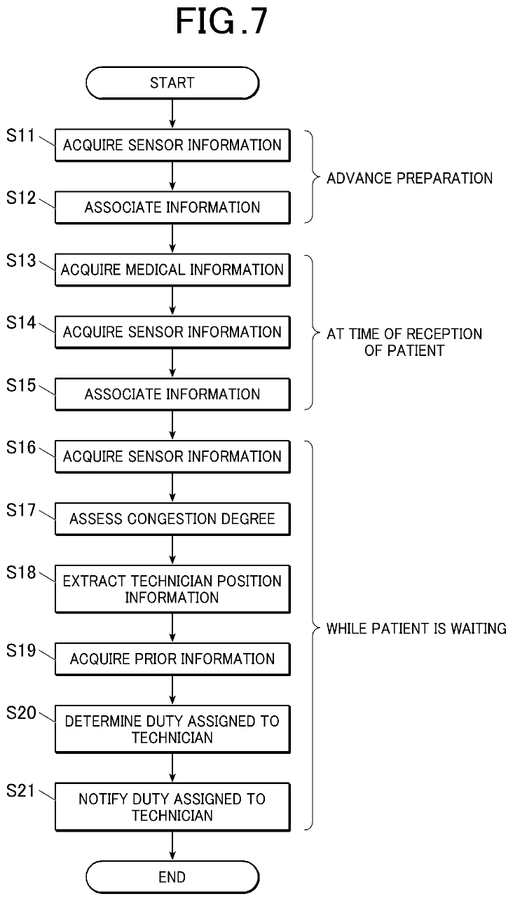

Next, the support process in the examination work support apparatus 1 will be described with reference to FIG. 7.

In Case 2, determining a technician's duty is supported.

### (Advance Preparation)

First, the controller 11 acquires sensor information from the sensor section 16 (Step S11). Specifically, the controller 11 acquires optical camera images of technicians in the examination rooms from the sensor section 16.

Next, the controller 11 associates prior information with the sensor information (Step S12). Specifically, the controller 11 associates the optical camera image of each technician with the prior information on the technician.

### (At Time of Reception of Patient)

Since Steps S13 to S15 are the same as Steps S1 to S3, descriptions thereof will be omitted.

### (While Patient is Waiting)

Next, the controller 11 acquires sensor information from the sensor section 16 (Step S16; acquisition step), and identifies the patient. Specifically, the controller 11 acquires, from the sensor section 16B disposed in the waiting room, an optical camera image obtained while the patient is waiting and identifies the patient.

Next, the controller 11 counts the number of persons identified as patients in the sensor information, and assesses the congestion degree (Step S17). Specifically, the controller 11 counts the number of persons identified as patients captured in the optical camera image, and determines that the congestion degree is high when the number thereof exceeds a predetermined value. In a case where waiting rooms correspond to examinations, the above leads to assessment of the congestion degrees for examinations. Even if waiting rooms do not correspond to examinations, the controller 11 can determine which examination has a high congestion degree by referring to medical information on patients.

Next, the controller 11 extracts technician position information on each technician from the sensor information (Step S18; extraction step). In Step S18, the operating state of each technician can be acquired from the technician position information on the technician. Specifically, the controller 11 determines where a technician is based on outputs from the sensor section 16 disposed in each room exemplified by the reception area, the waiting room, the examination rooms and the control room, and acquires the position information.

Next, the controller 11 acquires prior information from the storage section 14 (Step S19; acquisition step). Specifically, the controller 11 acquires the prior information including examination information indicating examinations performable by each technician.

Next, the controller 11 determines, as support information, duties assigned to technicians (Step S20).

More specifically, if the congestion degree for general radiography is low and the congestion degree for computed tomography (CT) is high, the controller 11 assigns, among the technicians in charge of general radiography, a technician capable of performing CT examination to perform the CT examination. In this case, the operating states of the technicians may also be taken into consideration.

Note that the determination method may involve trying all assignment patterns to make the determination, or learning an optimal combination in advance and performing inference processing using machine learning.

If, in Step S19, as the prior information, examination room information indicating which examination(s) is performable in an examination room and examination information indicating a time required for an examination are acquired, a technician in charge of general radiography but capable of performing CT examination may be promptly assigned to a CT examination room in which only examinations performable by the technician are performed.

As in Case 1, the technician assignment may be determined such that the examination end time is the earliest.

Next, the controller 11 causes the display part 15 to notify the duties assigned to the technicians (Step S21; notification control step), and ends the support process.

In this manner, the efficiency of the examination work can be improved more effectively.

More specifically, by optimizing the technician assignment, delays in examinations due to a shortage of technicians during congestion or at the timing when assistance for a patient is required are prevented, thereby improving examination efficiency.

### Support Process: Case 3

The above-described case 1 and case 2 may be combined.

In this case, in the support process, the patient assignment and the technician assignment are determined so as to make the examination end time earliest, based on information on examinations that can be performed by each combination of an examination room and a technician, information on a time required for each examination, the position and attribute of a patient(s), and the congestion state (of each waiting room and/or each examination).

As in Case 1 and Case 2, in making the determination, all combinations may be tried, or machine learning may be used.

### Others

In the above description, an optical camera image is used as the sensor information; however, the present disclosure is not limited to this example.

For example, radio frequency identification (RFID) may be used as the sensor information.

### Advantageous Effects

As described above, the examination work support apparatus 1 includes: the acquisition section (controller 11) that acquires medical information, sensor information acquirable in real time, and prior information; the extraction section (controller 11) that extracts, based on the information acquired by the acquisition section, one or more of patient position information on a patient, patient attribute information on the patient, and medical professional position information on a medical professional; the determination section (controller 11) that determines, based on the information acquired by the acquisition section and the information extracted by the extraction section, support information on one or more of an examination order number for the patient, an examination room, and a duty assigned to the medical professional; and the notification controller (controller 11) that causes the notifier to notify the support information determined by the determination section.

Accordingly, the efficiency of examination work can be improved more effectively.

Further, the examination work support system 100 includes: the acquisition section (controller 11) that acquires medical information, sensor information acquirable in real time, and prior information; the extraction section (controller 11) that extracts, based on the information acquired by the acquisition section, one or more of patient position information on a patient, patient attribute information on the patient, and medical professional position information on a medical professional; the determination section (controller 11) that determines, based on the information acquired by the acquisition section and the information extracted by the extraction section, support information on one or more of an examination order number for the patient, an examination room, and a duty assigned to the medical professional; and the notification controller (controller 11) that causes the notifier to notify the support information determined by the determination section.

Accordingly, the efficiency of examination work can be improved more effectively.

Further, the examination work support method that is performed by the computer of the examination work support apparatus 1 includes: the acquisition step (Steps S1, S2, S4, S7, S11, S13, S14, S16, S19) of acquiring medical information, sensor information acquirable in real time, and prior information; the extraction step (Steps S5, S6, S18) of extracting, based on the information acquired by the acquisition step, one or more of patient position information on a patient, patient attribute information on the patient, and medical professional position information on a medical professional; the determination step (Step S8, S20) of determining, based on the information acquired by the acquisition step and the information extracted by the extraction step, support information on one or more of an examination order number for the patient, an examination room, and a duty assigned to the medical professional; and the notification control step (Steps S9, S21) of causing the notifier to notify the support information determined by the determination step.

Accordingly, the efficiency of examination work can be improved more effectively.

Further the program causes the computer of the examination work support apparatus 1 to function as: the acquisition section (controller 11) that acquires medical information, sensor information acquirable in real time, and prior information; the extraction section (controller 11) that extracts, based on the information acquired by the acquisition section, one or more of patient position information on a patient, patient attribute information on the patient, and medical professional position information on a medical professional; the determination section (controller 11) that determines, based on the information acquired by the acquisition section and the information extracted by the extraction section, support information on one or more of an examination order number for the patient, an examination room, and a duty assigned to the medical professional; and the notification controller (controller 11) that causes the notifier to notify the support information determined by the determination section.

Accordingly, the efficiency of examination work can be improved more effectively.

Although the present disclosure has been described in detail above with reference to the embodiment, the present disclosure is not limited to the above-described embodiment, and various modifications may be made without departing from the scope of the present disclosure.

For example, the above description discloses an example in which a hard disk, a semiconductor nonvolatile memory, or the like is used as a computer-readable medium storing the program according to the present disclosure; however, the present disclosure is not limited to this example. As another computer-readable medium, a portable recording medium such as a CD-ROM may be used.

In addition, detailed configurations and operations of the respective apparatuses may be appropriately modified without departing from the scope of the present disclosure.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. An examination work support apparatus comprising a hardware processor that
acquires medical information, sensor information acquirable in real time, and prior information,
extracts, based on the acquired information, one or more of patient position information on a patient, patient attribute information on the patient, and medical professional position information on a medical professional,
determines, based on the acquired information and the extracted information, support information on one or more of an examination order number for the patient, an examination room, and a duty assigned to the medical professional, and
causes a notifier to notify the determined support information.

2. The examination work support apparatus according to claim 1, wherein the hardware processor determines the support information based on a viewpoint of minimization of a waiting time of the patient and/or maximization of a number of examinations per unit time.

3. The examination work support apparatus according to claim 1, wherein the hardware processor acquires the patient position information and/or the medical professional position information from an image that is the sensor information.

4. The examination work support apparatus according to claim 1, wherein the medical information is information stored in a radiology work management system.

5. The examination work support apparatus according to claim 1, wherein the prior information includes standard examination time information for each of the attribute information, for each examination, and for each of the medical professional.

6. The examination work support apparatus according to claim 1, wherein the prior information includes performable examination information for each of the examination room and for each of the medical professional.

7. The examination work support apparatus according to claim 1, wherein the hardware processor assesses a congestion degree of a waiting room based on the patient position information.

8. The examination work support apparatus according to claim 7, wherein the hardware processor switches necessity and unnecessity of a process of the determination or switches a level of the process of the determination, based on the congestion degree.

9. An examination work support system comprising a hardware processor that
acquires medical information, sensor information acquirable in real time, and prior information,
extracts, based on the acquired information, one or more of patient position information on a patient, patient attribute information on the patient, and medical professional position information on a medical professional,
determines, based on the acquired information and the extracted information, support information on one or more of an examination order number for the patient, an examination room, and a duty assigned to the medical professional, and
causes a notifier to notify the determined support information.

10. An examination work support method that is performed by a computer of an examination work support apparatus, comprising:
acquiring medical information, sensor information acquirable in real time, and prior information;
extracting, based on the acquired information, one or more of patient position information on a patient; patient attribute information on the patient, and medical professional position information on a medical professional;
determining, based on the acquired information and the extracted information, support information on one or more of an examination order number for the patient, an examination room, and a duty assigned to the medical professional; and
causing a notifier to notify the determined support information.
